(19) United States
(12) Patent Application Publication
(10) Pub. No.: US 2015/0126828 A1
(43) Pub. Date: May 7, 2015

(54) BLOOD SAMPLING

(71) Applicant: IBIS BIOSCIENCES, INC., Carlsbad, CA (US)

(72) Inventors: Steven A. Hofstadler, Vista, CA (US); David J. Ecker, Encinitas, CA (US); Christopher C. Sappenfield, San Diego, CA (US)

(21) Appl. No.: 14/514,920

(22) Filed: Oct. 15, 2014

Related U.S. Application Data
(60) Provisional application No. 61/891,180, filed on Oct. 15, 2013.

(51) Int. Cl. A61B 5/15; G01N 33/49; A61B 5/145; A61B 5/157; A61B 5/00; C12Q 1/68; A61B 5/153

(52) U.S. Cl. CPC A61B 5/150755; C12Q 1/6806; G01N 33/491; A61B 5/153; A61B 5/150992; A61B 5/157; A61B 5/4839; A61B 5/14546
USPC 600/309; 600/573; 600/576

(57) ABSTRACT

Provided herein is technology relating to sampling blood and particularly, but not exclusively, to methods, devices, and systems for high-efficiency isolation of analytes from blood, e.g., for sensitive detection of analytes present in blood at low concentrations.

### CROSS-REFERENCE TO RELATED APPLICATIONS

The present Application claims priority to U.S. Provisional Application Ser. No. 61/891,180 filed 15 Oct. 2013, the entirety of which is incorporated by reference herein.

### FIELD OF INVENTION

Provided herein is technology relating to sampling blood and particularly, but not exclusively, to methods, devices, and systems for high-efficiency isolation of analytes from blood, e.g., for sensitive detection of analytes present in blood at low concentrations.

### BACKGROUND

Blood samples are often obtained from patients for assessing analytes associated with a patient's physiological and/or biochemical status, e.g., to provide data relevant to disease, mineral content, drug effectiveness, and/or organ function. In some cases, medical data are collected by qualitative and/or quantitative detection in the patient's blood of an analyte that is a disease-associated entity such as a pathogen.

Conventional practices for collecting a blood sample use finger pricks and heel sticks to collect minute quantities of blood and venipucture for collection of larger blood samples, typically in 3-10 milliliter vacuum tubes. Collection of a 3-10 milliliter sample represents approximately 0.1% to 0.2% of the total blood volume in an adult human. As such, analytes present in extremely low concentration in the blood may not be present in conventional samples, may be present below a detectable or useful threshold for analysis, or may be masked by human DNA or other biomolecules that are not analytically relevant for the desired test.

The sensitivity of some diagnostic assays, e.g., for blood borne pathogens, is limited by the volume of blood sampled. Consequently, particular problems are associated with the detection of some pathogens in a patient's blood. First, the number of a free-flowing pathogen (e.g., bacteria) in blood can be extremely low, e.g., as low as 1-3 colony forming units (CFU) per milliliter of blood. At this concentration, a single 3-10 milliliter sample drawn from a patient will contain few or no bacteria and thus does not provide a sample that can provide meaningful results. In addition, some microbes (e.g., bacteria) associate in clumps and are thus not uniformly distributed throughout the patient's blood. Both the low concentration and uneven distribution lead to stochastic sampling errors and false negative results associated with the small sample size relative to the patient's total blood volume.

Some solutions to this problem involve drawing a larger blood volume as a sample. However, such an approach has drawbacks—for instance, obtaining larger sample volumes may place additional stress on some patients, such as children, the elderly, or the chronically ill. In addition, larger samples also contain increased amounts of non-analyte substances. For example, a blood sample contains approximately 5-15 million white blood cells per milliliter and approximately 60-180 micrograms of subject DNA per milliliter. At these concentrations, subject (e.g., human) DNA co-extracted with pathogen DNA in a total DNA extraction from a blood sample is present at high amounts, e.g., at amounts of more than 10 micrograms per reaction, that are known to inhibit PCR assays. Methods to extract and/or enrich bacterial DNA preferentially from other DNA (e.g., human DNA) have proven to be unsatisfactory for some detection assays for pathogens and, in addition, such extraction methods are often prone to low levels of bacterial contamination. New sampling approaches are needed to analyze large volumes of blood with sensitive detection assays without putting a patient at risk.

### SUMMARY

Accordingly, provided herein is technology for collecting an analyte from blood for analysis. Aspects of the technology relate to a device for collecting an analyte (e.g., a pathogen such as one or more bacteria) from blood. In some embodiments, the device is a vascular implant that captures analytes (e.g., free circulating bacteria) from blood diverted from the patient through the device (e.g., implanted within the radial/ulnar vasculature). In some embodiments, the device provides a high surface-to-volume ratio, short-term vascular implant for collecting (e.g., concentrating) analytes such as pathogens from blood. In some embodiments, the device comprises a capture surface (e.g., a capture matrix) with a high affinity for a broad range of analytes, pathogens, microbes, viruses, etc. In some embodiments, the device comprises a capture surface with a high affinity for a specific organism or group of organisms. In some embodiments, the technology provides an extracorporeal device for collecting an analyte. The technology also provides related methods and systems for collecting an analyte from blood and testing the analyte. The technology is useful to provide analytes for a broad suite of assays, e.g., molecular diagnostics related to nucleic acid amplification (e.g., real time PCR), and as a sampling technique for down-stream culture applications. The technology is compatible with rapid analytical platforms such as mass spectrometry (e.g., MALDI-TOF, etc.) and antibody-based detection approaches, microarrays, and immunofluorescent methods. The technology finds use in clinics, hospitals, military medicine, veterinary, and/or emergency contexts.

Total blood flow through a human arm at rest is approximately 500 milliliters per minute. As such, a 30-minute collection with embodiments of the device would effectively sample 15 liters of patient blood. As an example, assuming a capture efficiency of at least 1%, the sampling would provide at least a 3-fold improvement in analyte sampling relative to a 5-milliliter static blood draw into a vacuum tube. Capture efficiencies greater than 1% provide (e.g., proportionally) greater improvements over the conventional technology. A typical adult has a blood volume of approximately 5 liters. By a calculation similar to that above, an 8-hour (e.g., overnight) collection with the technology provided herein samples the entirety of a patient's total blood volume multiple times. For example, assuming a capture efficiency of at least 1%, an 8-hour sampling would sample the equivalent of at least 24 liters of blood or approximately 5 times the total blood volume of a normal adult human. As such, the technology overcomes the stochastic sampling limitations associated with small (˜5 milliliter) samples taken from a patient using conventional technology.

As such, embodiments of the technology provide a short-term vascular implant device for collecting an analyte from blood (e.g., diverted from a patient's blood flow), the device comprising a capture matrix and a tubing component. In some embodiments, the capture matrix is removable from the device and in some embodiments the device further comprises a removable cartridge comprising the capture matrix. The device captures analyte from patient blood and thus has a higher affinity and/or specificity for the analyte than for a component of the patient's blood. Accordingly, in some embodiments the capture matrix has a higher affinity for the analyte relative a component of the patient's blood.

Some embodiments of the technology provide a short-term vascular implant device for collecting an analyte from blood, the device consisting of or consisting essentially of a capture matrix, a tubing component, and a removable cartridge comprising a resin bound oligo-acyl-lysine. Some embodiments of the technology provide a short-term vascular implant device for collecting an analyte from blood, the device consisting of or consisting essentially of a capture matrix, a tubing component, and a removable cartridge.

Some embodiments of the technology provide a short-term vascular implant device for collecting an analyte from blood, the device consisting of or consisting essentially of a capture matrix, a tubing component comprising a first venipuncture tip and a second venipuncture tip, a removable cartridge comprising a resin bound oligo-acyl-lysine, and an anti-thrombotic agent. Some embodiments of the technology provide a short-term vascular implant device for collecting an analyte from blood, the device consisting of or consisting essentially of a capture matrix; a tubing component comprising a first venipuncture tip and a second venipuncture tip; a removable cartridge comprising a resin bound oligo-acyl-lysine; and a filter, valve, cartridge interface, or Y-connector.

Some embodiments of the technology provide a short-term vascular implant device for collecting an analyte from blood, the device consisting of or consisting essentially of a removable capture matrix and a tubing component. Some embodiments of the technology provide a short-term vascular implant device for collecting an analyte from blood, the device consisting of or consisting essentially of a removable capture matrix comprising a resin bound oligo-acyl-lysine and a tubing component.

The device is not limited in the analyte captured from the patient blood. For example, in some embodiments the analyte is a pathogen (e.g., one or more bacteria, eukaryotes, archaea, and/or viruses). In some embodiments the analyte is a nucleic acid. Embodiments of the technology relate to the device before and after finding use in capturing an analyte. Accordingly, in some embodiments the device further comprises the analyte (e.g., one or more concentrated analyte(s)).

The technology is not limited in the material and/or composition of the capture matrix. For example, in some embodiments, the capture matrix comprises a resin bound oligo-acyl-lysine.

Blood is diverted into the device from the patient's circulatory system, e.g., by inserting the device into a blood vessel of the patient. In some embodiments, the device is an extracorporeal device further comprising components for venipuncture, e.g., the device further comprises a first venipuncture tip and a second venipuncture tip. In addition, some embodiments provide for a device comprising a biologically active compound for elution into the blood (e.g., for local and/or systemic delivery of the biologically active compound) and/or to minimize thrombogenesis, imflammation, and/or patient discomfort. As such, some embodiments provide a device comprising an anti-thrombogenic agent. The device may comprise other components and features to aid a user in using the device. For example, in some embodiments the device comprises a filter (e.g., to prevent emboli from entering the patient blood stream), a valve (e.g., to slow or stop blood flow through the device to change a cartridge, withdraw a sample, extract the capture matrix, etc.), a cartridge interface (a feature for attaching cartridges to the device), or a Y-connector (e.g., for extracting samples and/or for delivering a solution into the bloodstream). Certain types of blood flow behavior are preferred in the device, e.g., to minimize turbulent flow and other physical disturbances that can damage blood components or disrupt flow. Accordingly, in some embodiments a device is provided that is configured to promote laminar flow of blood through the device.

In addition, described herein are embodiments of methods for capturing an analyte from a patient's blood, the methods comprising diverting patient blood through a short-term vascular implant device as described herein and capturing an analyte from the patient's blood on the capture matrix. Further method embodiments provide a step of recovering the analyte from the capture matrix. Continuous collection of analytes from patient blood can occur for various lengths of time, e.g., from minutes to hours. As such, in some embodiments the diverting is over a time of 5 minutes to 12 hours or any time increment therebetween (e.g., 5 to 60 minutes, 1 to 12 hours). During this time, patient blood is continuously sampled and analyte captured during the exposure of the capture matrix to the blood. Thus, in some embodiments the capture matrix is exposed to more than 5 milliliters of patient blood, more than 500 milliliters of patient blood, more than 1 liter of patient blood, more than 5 liters of patient blood, more than 10 liters of patient blood, more than 20 liters of patient blood, more than 0.5× the total blood volume of the patient, more than lx the total blood volume of the patient, more than 2× the total blood volume of the patient, more than 3× the total blood volume of the patient, more than 4× the total blood volume of the patient, or more than 5× the total blood volume of the patient. After capture, some method embodiments provide a step of analyzing the analyte, e.g., using a quantitative and/or a qualitative assay. In addition, medical treatment decisions can be made based on the test results. As such, in some embodiments the methods further comprise administering a drug to the patient based on the result of the analyzing.

Related kit embodiments provide a kit for the analysis of a patient blood sample for an analyte, the kit comprising a short-term vascular implant device as described herein and a reagent for processing a captured analyte. The kits may be used to capture bacterial (or other) non-patient cells from patient blood; as such, some embodiments comprise reagents for lysing bacterial cells to produce a cell lysate and preparing nucleic acid from the cell lysate. In some embodiments of kits, the kits comprise a device as described herein and one or more cartridges comprising a capture matrix. Furthermore, some kit embodiments comprise reagents for analyzing an analyte or a sample prepared from the analyte. For example, in some embodiments the kits comprise reagents for polymerase chain reaction analysis, mass spectrometry analysis, or immunological analysis of a captured analyte.

In some embodiments, the technology comprises use of a detection device, e.g., to detect, quantify, characterize, and/or modify (e.g., analyze) the captured analyte. Examples of a detection device include, but are not limited to, a thermocycler (e.g., a real-time PCR apparatus), a melting temperature apparatus, a calorimeter, a nucleic acid sequencer, a culture substrate or medium, a mass spectrometer, a chromatographic instrument (e.g., HPLC), a fluorimeter, a spectrometer (UV-vis, IR, Raman), an atomic absorption apparatus, an immunological apparatus, a microscope, a scale, a nuclear magnetic resonance apparatus, a device to measure turbidity, a densitometer, a density gradient, surface plasmon resonance, etc. In some embodiments, dyes, primers, probes, antibodies, aptamers, enzymes, and the like are associated with the analyte and detection device for analysis of the analyte.

Some embodiments comprise a processor (e.g., a microprocessor) configured to perform instructions (e.g., as provided in software, firmware, etc.) for performing analysis of data acquired from analysis (measurement, etc.) of the analyte. Some embodiments comprise data storage, transmission, and display capabilities. Some embodiments comprise the input of data from analysis of an analyte and output to the user an actionable result, e.g., based on a calculation performed by the processor as instructed by the software.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1A is a drawing showing a vascular implant device embodiment of the technology.

FIG. 1B is a drawing showing an exemplary placement of a vascular implant device embodiment of the technology.

FIG. 2A is a drawing showing an extracorporeal device embodiment of the technology.

FIG. 2B is a drawing showing an exemplary placement of an extracorporeal device embodiment of the technology.

FIG. 3 is a chemical structure of a resin bound oligo-acyl-lysine capture matrix. L represents a generic linker moiety and the rectangle represents a generic solid support.

FIG. 4 is a drawing showing the capture of an analyte from blood using a device embodiment of the technology and/or an associated method embodiment.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to sampling blood and particularly, but not exclusively, to methods, devices, and kits for high-efficiency isolation of analytes from blood, e.g., for sensitive detection of analytes present in blood at low concentrations. In the description of the technology, the section headings used herein are for organizational purposes only and are not to be construed as limiting the described subject matter in any way. Furthermore, in this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The terms “bacteria” and “bacterium” refer to prokaryotic organisms of the domain Bacteria in the three-domain system (see Woese C R, et al., Proc Natl Acad Sci U SA 1990, 87: 4576-79). It is intended that the terms encompass all microorganisms considered to be bacteria including Mycobacterium, Mycoplasma, Chlamydia, Actinomyces, Streptomyces, and Rickettsia. All forms of bacteria are included within this definition including cocci, bacilli, spirochetes, spheroplasts, protoplasts, etc. In some embodiments, bacteria are capable of causing disease and product degradation or spoilage.

As used herein, a “pathogen” is an organism or agent that is capable of causing a disease. The terms “non-pathogenic microbe” or “non-pathogenic microorganism” include all known and unknown non-pathogenic microbes (Bacteria, Archaea, and/or Eukarya) and any pathogenic microbe that has been mutated or converted to a non-pathogenic state. Furthermore, a skilled artisan recognizes that some microbes may be pathogenic to specific species and non-pathogenic to other species; thus, these microbes can be utilized in the species in which it is non-pathogenic or mutated so that it is non-pathogenic. One of skill in the art also recognizes that some pathogens can switch states such that they are at times pathogenic to a species and at other times not pathogenic to the same species.

As used herein, the terms “culture” and “cell culture” refer to any in vitro culture of cells, including, e.g., prokaryotic cells and eukaryotic cells. Included within this term are continuous cell lines (e.g., with an immortal phenotype), primary cell cultures, transformed cell lines, finite cell lines (e.g., non-transformed cells), bacterial or archaeal cultures in or on solid or liquid media, and any other cell population maintained in vitro.

As used herein, the terms “subject” and “patient” are used interchangeably to describe an animal, including mammals, to which the present technology is/are applied. Mammalian species that benefit from the disclosed technologies include, but are not limited to, apes, chimpanzees, orangutans, humans, and monkeys; domesticated animals (e.g., pets) such as dogs, cats, guinea pigs, and hamsters; veterinary uses for large animals such as cattle, horses, goats, and sheep; and any wild animal for veterinary or tracking purposes.

As used herein, the terms “surgeon” or “physician” are merely for literary convenience. The terms should not be construed as limiting in any way. The devices, apparatuses, methods, techniques and/or procedures of the technology described could be utilized by any person desiring or needing to do so and having the necessary skill and understanding of the technology.

Also, as used herein, and unless otherwise specifically stated, the terms “operable communication” and “operably connected” mean that the particular elements are connected in such a way that they cooperate to achieve their intended function or functions. The “connection” may be direct, or indirect, physical or remote.

In addition, references to “first”, “second”, and the like (e.g., first and second tips of the device), as used herein, and unless otherwise specifically stated, are intended to identify a particular feature of which there are at least two. However, these references are not intended to confer any order in time, structural orientation, or sidedness (e.g., left or right) with respect to a particular feature.

### Aspects of the Technology

Although the disclosure herein refers to certain illustrated embodiments, it is to be understood that these embodiments are presented by way of example and not by way of limitation.

### Devices

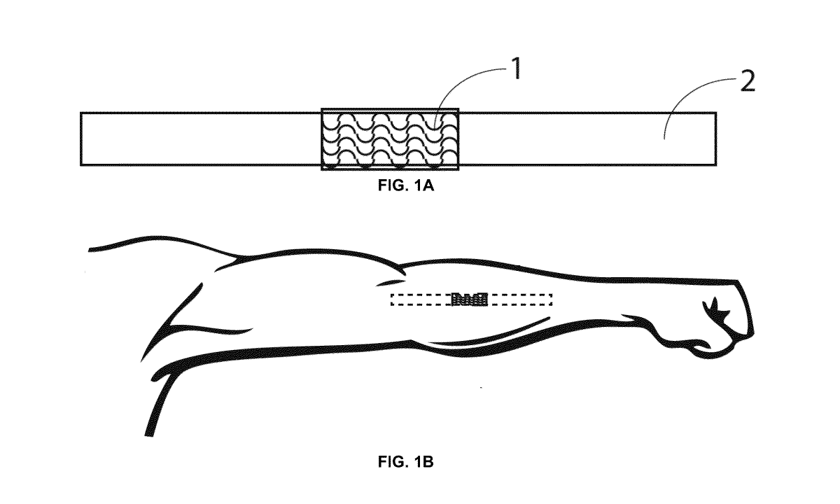

In some embodiments, the technology provides a vascular implant device (e.g., short-term) for continuous-flow sampling of patient blood (FIG. 1). An embodiment of the device comprises a capture matrix 1 and a tubing component 2 (e.g., comprising an inflow component and an outflow component). The capture matrix 1 is removable from the tubing component to provide for the recovery of the collected analyte from the capture matrix, e.g., for downstream analysis. In some embodiments, the device measures less than a meter, less than 50 cm, less than 40 cm, less than 30 cm, less than 20 cm, less than 10 cm, or less than 5 cm in length (from the first and second ends of the tubing component). As such, in some embodiments, the device can be held in the hand and transported and/or administered by a medical professional. In addition, in some embodiments the device is attached to a patient without restricting the patient's mobility.

In some embodiments, a removable cartridge comprises the capture matrix. The removable cartridge is attached to the tubing component 2 when the device is used to collect an analyte from blood. The removable cartridge is detached from the tubing component 2 to provide access to the capture matrix 1 to provide for the recovery of the collected analyte from the capture matrix 1, e.g., for downstream analysis. In some embodiments, the cartridge is operatively connected to the tubing component 2 using a suitable connector, e.g., a luer taper (e.g., as defined by ISO 594, DIN 1707:1996, and EN 20594-1:1993) such as a luer-lock or a luer-slip connector, snap joint, threaded or barbed mated male and female connectors, etc. In some embodiments, the cartridge is operatively connected to the tubing component 2 by an intermediate tube, connector, etc. that operably links the cartridge to the tubing component 2. In some embodiments, the device comprises one or more cartridge interfaces to which one or more cartridges are attached. FIG. 1B shows an exemplary placement of the device within the radial/ulnar vasculature in the arm of a patient.

Some embodiments provide a variety of modular cartridges comprising a variety of different capture matrices such that the different cartridges are capable of being attached to the device (e.g., the tubing component), e.g., to provide for the collection of different analytes by attaching different cartridges to the device. Embodiments provide for attaching a single cartridge to the device and for attaching multiple cartridges in series to the device. In some embodiments, a cartridge comprises one capture matrix and in some embodiments a cartridge comprises more than one capture matrix (e.g., for the capture of more than one analyte by a single cartridge).

In some embodiments, the device comprises a peripheral cannula. For example, in some embodiments, the device comprises a peripheral intravenous line comprising a short catheter (e.g., 1, 2, 3, 4, or 5 centimeters long) inserted through the skin into a peripheral vein. In some embodiments, the device comprises a cannula-over-needle device in which a flexible plastic cannula is mounted on a metal trocar. Once the tip of the needle and cannula are located in the vein the trocar is withdrawn and the cannula advanced inside the vein to the appropriate position and secured.

In some embodiments, the device comprises a filter to prevent any emboli formed within the device from entering the patient's bloodstream.

In some embodiments, it is advantageous to engineer the device to have a form factor that minimizes turbulent flow and other physical phenomena that promote thrombosis and/or damage to blood components. Accordingly, in some embodiments design of the device is informed by knowledge of the characteristics of hydrodynamic blood flow through the vasculature, through biomedical devices, and through interfaces between the vasculature and biomedical devices (in-flow and out-flow). Blood has complicated rheology and there are many models to describe it on different scales. For example, in some models, blood flow can be described by the classic Navier-Stokes equations. More complicated blood-flow models are implemented with computer modeling, e.g., using the software packages Fluent, CFX (both from ANSYS, Inc., Canonsburg, Pa.), STAR-CD (CD-Adapco, Melville, N.Y.), AcuSolve (ACUSIM Software, Mountain View, Calif.), and Adina (Adina R&D, Watertown, Mass.).

In some models, blood passing through the device is pulsating blood and can be assumed to have the properties of a Newtonian fluid (e.g., an incompressible fluid having a laminar flow) at a shear rate greater than 100 s⁻¹, e.g., above 1000 s⁻¹ (see, e.g., Cokelet, “The Rheology and Tube Flow of Blood”, Chapter 14 in Skalak et al. (eds.), Handbook of Bioengineering (McGraw-Hill, New York, 1987), hereby incorporated by reference herein in its entirety). At these shear rates, blood viscosity is constant. Whole blood with normal hematocrit (approximately 45%) has a viscosity of about 4.2 cP at 37° C., which is about 1.8 times the viscosity of water at the same temperature (see, e.g., Schneck, “Cardiovascular Mechanics”, Chapter 10 in Enderle et al. (eds.), Introduction to Biomedical Engineering (Academic Press, New York, 2000), hereby incorporated by reference herein in its entirety). Protoytpe devices can be modeled in silico and ex vivo for testing their hydrodynamic properties.

In some embodiments, the device does not comprise one or more of a pump, a pressure monitor, a dialyzer, a check valve, an electronic component, and/or a component requiring electric power (e.g., an alternating or direct current and/or voltage).

In some embodiments, a portion of the device that contains the capture matrix is marked for easy identification. In some such embodiments, the device is accessed and a capture matrix is removed and replaced by a new capture matrix. In some embodiments, the removal involves removal of the device in its entirety from a subject. In some embodiments, at least a portion of the device remains in the subject while the capture matrix is removed and/or replaced.

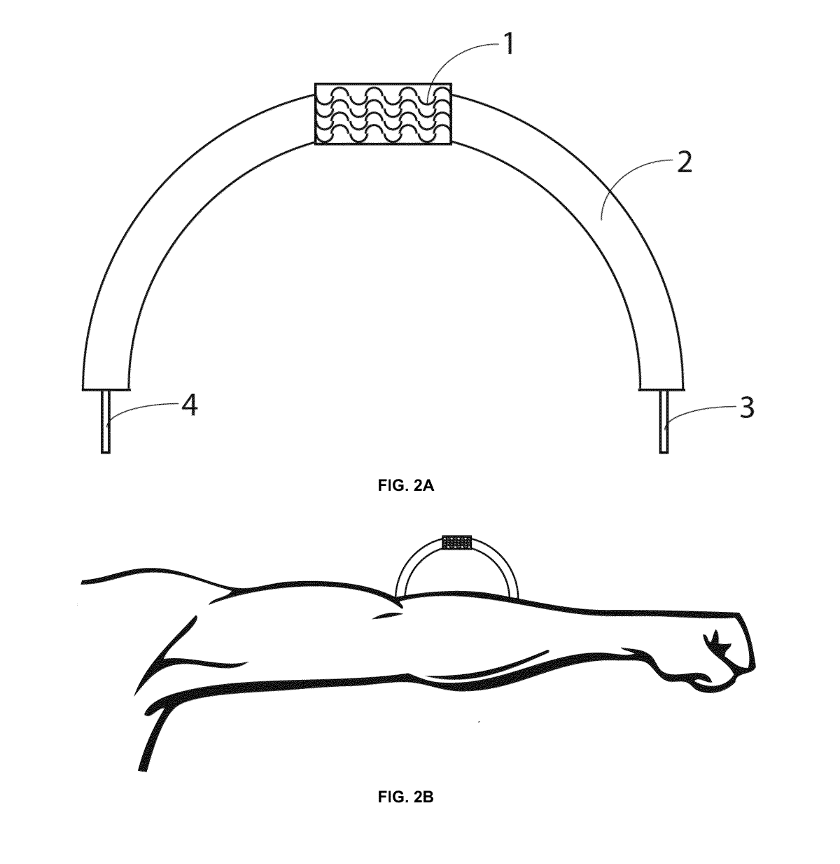

Some embodiments provide a minimally invasive, extracorporeal device for continuous-flow sampling of patient blood, e.g., as shown in FIG. 2A. In embodiments related to an extracorporeal device, the device comprises a capture matrix 1, a tubing component 2 (e.g., comprising an inflow component and an outflow component), a first tip 3 for venipuncture, and a second tip 4 for venipuncture. The capture matrix 1 is removable from the tubing component 2 to provide for the recovery of the collected analyte from the capture matrix 1, e.g., for downstream analysis. In some embodiments, the device measures less than a meter from the first tip 3 to the second tip 4. In some embodiments, the device measures less than 50 cm, less than 40 cm, less than 30 cm, less than 20 cm, less than 10 cm, or less than 5 cm from the first tip 3 to the second tip 4. As such, in some embodiments, the device can be held in the hand and transported and/or administered by a medical professional. In addition, in some embodiments the device is attached to a patient without restricting the patient's mobility.

In extracorporeal embodiments, the first tip 3 and the second tip 4 (collectively “tips”) may be the same or different in material, shape, structure, design, etc. In some embodiments, the tips comprise components that are the same or similar to conventional intravenous access devices. For example, in some embodiments the tip is a hollow needle, e.g., a hypodermic needle. The caliber of a cannula or needle is commonly indicated in “gauge”, with 14 being a very large cannula (e.g., as used in resuscitation settings) and a gauge of 24 to 26 being the smallest typically used in medicine. The most common sizes are 16-gauge (e.g., a midsize line used for blood donation and transfusion), 18-gauge and 20-gauge (e.g., an all-purpose line for infusions and blood draws), and 22-gauge (e.g., an all-purpose pediatric line). Gauges of 12 and 14 as used in peripheral lines are capable of delivering large volumes of fluid extremely fast, e.g., for emergency medicine. These lines are frequently called “large bores” or “trauma lines”. The tips are operatively connected to the tubing component 2 using a suitable connector, e.g., a luer taper (e.g., as defined by ISO 594, DIN 1707:1996, and EN 20594-1:1993) such as a luer-lock or luer-slip connector, snap joint, threaded or barbed mated male and female connectors, etc. In some embodiments, the tips are operatively connected to the tubing component 2 by an intermediate tube, connector, etc. that operably links one of more of the tips to the tubing.

FIG. 2B shows an exemplary placement of the extracorporeal device in the arm of a patient. The first tip 3 and the second tip 4 are inserted into a patient's vasculature such that some of the patient's blood is routed through the device to contact the capture matrix. In some embodiments, the device is configured for addition inline to an in-line blood diverting apparatus such as a dialysis machine, a blood warmer, a blood chiller, a blood conditioner, a blood treatment apparatus, a blood filtration apparatus, a blood oxygenator, a blood pump, a heart and/or cardiopulmonary bypass system, or a blood air removal system, e.g., to add functionality to the in-line blood diverting apparatus such as isolating an analyte from blood.

### Materials

In various embodiments, the components of the device comprise one or more materials. Generally, the device is designed to be biocompatible with the patient by using biocompatible materials for the device components that contact a patient (e.g., patient blood and/or other tissue) or biological substances (e.g., blood) flowing from the patient through the device. In particular, the biocompatibility of a medical device that is inserted within the cardiovascular system for transient diagnostic or therapeutic purposes refers to the ability of the device to carry out its intended function in contact with flowing blood and/or patient tissue (e.g., skin, muscle, etc.), with minimal interactions between the device and patient tissue (e.g., blood) that adversely affects device performance and without inducing uncontrolled activation of cellular or plasma protein cascades. For example, it is advantageous in some embodiments that the device does not comprise a thrombogenic material, e.g., a material that produces adverse reactions when placed in contact with blood such as formation of a clot or thrombus, shedding or nucleation of emboli (detached thrombus), destruction of circulating blood components, and/or activation of the complement system (and associated inflammation responses) and other immunologic pathways. In some embodiments, materials are chosen that minimize and/or eliminate adsorption of blood proteins, blood cells, platelets, and other blood components to surfaces. In some embodiments, the materials comprise polyurethane (PU) (e.g., Biomer, Pellethane, Mitrathane, Tecoflex), polyethyleneoxide (PEO), silicone rubber, and/or polytetrafluoroethylene (PTFE) (e.g., Impra, Goretex, Vitagraft). In some embodiments, the material is polyvinylchloride, polyethylene, polystyrene, polyethylene-terephthalate (Dacron), polyamine, cellulose, dextran, polyacrylonitrile, polymethylmethacrylate, polysulfone, celluose acetate, and/or polydimethylsiloxane. Materials that are reactive to platelets in certain contexts include polystyrene (PS), polyvinylchloride (PVC), polyethylene, and polymethylmethacrylate (PMMA).

In some embodiments, the materials have a hydrophobic surface; in some embodiments, the materials have a hydrophilic surface; and, in some embodiments, the materials comprise an alternation of hydrophobic and hydrophilic motifs on a polymer surface (e.g., segmented polyurethane, bloc copolymers formed by alternating segments of polyurethanes (hydrophobic) and polyethers or polyesters (hydrophilic)). In some embodiments, a surface is modified to reduce interactions with blood components. In some embodiments, it is advantageous to use one material to provide desirable mechanical characteristics of the device or device component and to use a second material to modify the surface to provide desirable blood-interaction characteristics.

Accordingly, in some embodiments the device comprises biocompatible materials that are amenable to surface modification. In some embodiments, a surface is modified with a hydrogel, e.g., to improve the blood-compatibility of the device material. Examples of hydrogels for blood contact include but are not limited to poly(vinyl alcohol), polyacrylamides, poly(N-vinyl-2-pyrrolidone), poly(hydroxyethyl methacrylate), and poly(ethylene oxide). In some embodiments, a surface is modified with poly(ethylene glycol) (PEG); in some embodiments, a surface is modified with an albumin. In some embodiments, a surface incorporates fluorine or comprises a fluorine-containing compound.

Materials used for the venipuncture components such as the first tip 3 and the second tip 4 are generally the same as used for conventional hypodermic needles and cannulae (e.g., metals such as steel, stainless steel, carbon steel) used in conventional venipuncture. The material may be nickel plated to reduce or eliminate corrosion.

### Coatings

In some embodiments, one or more components of the device comprise a coating (e.g., are coated with a material). In particular embodiments the coating is a polymer, e.g., a polyester (lactide, glyatide, and e-caprolactone), cellules, poly(vinyl alcohol), PMMA, PBMA, povidone, poly(ethylene-co-vinyl alcohol), arabia rubber, bassora gum, EVAC, cellulose, or various other suitable compounds. In some embodiments, a coating comprises an additive. Suitable additives include cross-linking agents, dispersants (wetting agents), plasticizers, and, in some embodiments, a coating comprises the biologically active compounds described herein. In some embodiments, the function of the cross linking agent is to provide structural integrity to the coating, and cross-linking agents such as acylamine, amidoformate may be used. In some embodiments, the function of a dispersant (wetting agent) is to enhance dispersion of the polymer, to make the distribution of components of the solution more uniform, etc. In some embodiments, the function of the plasticizer is to improve the mechanical characteristics of the coating. Plasticizers including linear polymers such as polyaether are used in various embodiments.

### Biologically Active Compounds

In some embodiments, the device comprises a biologically active compound and/or releases a biologically active compound into the blood flowing through the device. For example, in some embodiments the device comprises an anti-thrombogenic agent to minimize and/or eliminate clotting within the device or in the patient's vasculature that may result from the use of the device, e.g., contact of patient's blood with device components that may promote a clotting response.

In some embodiments, the biologically active compound is an immunosuppressant compound, an anti-cancer agent, a hormone, an anti-inflammatory, an analgesic, an anti-anxiety agent, an anti-stenosis agent, etc. Suitable immunosuppressants include ciclosporinA (CsA), FK506, DSG(15-deoxyspergualin, 15-dos), MMF, rapamycin and its derivatives, CCI-779, FR 900520, FR 900523, NK86-1086, daclizumab, depsidomycin, kanglemycin-C, spergualin, prodigiosin25-c, cammunomicin, demethomycin, tetranactln, tranilast, stevastelins, myriocin, gloxin, FR 651814, SDZ214-104, bredinin, WS9482, and steroid. Suitable anti-thrombogenic drugs include heparin, aspirin, hirudin etc., GPIIb/IIIa receptor inhibitors such as tirofiban, eptifibatide, cilostazol, plavix, Ticlid, etc. Suitable anti-cancer agents include methotrexate, purine, pyridine, and botanicals (e.g. paclitaxel, colchicines, and triptolide), epothilone, antibiotics, and antibodies. Suitable additional anti-stenosis agents include batimastat, NO donor, 2-chlorodeoxyadenosine, 2-deoxycoformycin, FTY720, Myfortic, ISA (TX) 247, AGI-1096, OKT3, Medimmune, ATG, Zenapax, Simulect, DAB486-IL-2, Anti-ICAM-1, Thymoglobulin, Everolimus, Neoral, Azathipprine (AZA), Cyclophosphamide, Methotrexate, Brequinar Sodium, Leflunomide, Mizoribine.

### Capture Matrix

The device comprises a capture matrix to capture (e.g., collect and concentrate) one or more analytes from blood. The capture matrix comprises a material that provides for the recovery of the analyte or analytes from the capture matrix for downstream analysis. In some embodiments, the capture matrix does not lyse bacteria or other organisms that provide or comprise an analyte. In some embodiments, the capture matrix captures a type of biomolecule (e.g., nucleic acid) that is the analyte.

In some embodiments, the capture matrix comprises a biological or bio-mimetic material to capture one or more microbes, pathogens, bacteria, archaea, viruses, prions, and/or eukaryotes (e.g., fungi (e.g., yeasts), protozoa, etc). In some embodiments, the capture matrix is specific for a particular species, sub-species, strain, genus, or other taxonomic division of biological organisms or a biomolecule associated with and/or derived from a particular species, sub-species, strain, genus, or other taxonomic division of biological organisms. In some embodiments, the capture matrix is specific for a combination of multiple species, sub-species, genera, or other taxonomic divisions. In some embodiments, the capture matrix is specific for multiple organisms or groups of organisms not defined by taxonomy but by function, shape, composition (e.g., surface, (e.g., membrane) composition), size, reactivity, metabolic capacity, affinity for a capture entity (e.g., an antibody or antibodies), or other characteristics common to the group and/or defining the group (e.g., Gram negative, Gram positive). In some embodiments, the capture matrix is not specific to any particular organism and in some embodiments the capture matrix captures all organisms and/or biomolecules from a patient's blood except for patient cells and/or patient biomolecules. In some embodiments, the capture matrix comprises a solid support. The solid support capture matrix may be provided as a slurry, porous solid, packed column, etc., through which blood flows.

In some embodiments, the capture matrix is a fibrous material; in some embodiments, the capture matrix is a membrane. In some embodiments the capture matrix comprises a monofilament fiber, an eletrospun fiber, and/or a branched material. In some embodiments, blood flows through the capture matrix. In some embodiments, blood flows across the capture matrix (e.g., cross-flow or tangential flow filtration).

In some embodiments, the geometry of the capture matrix and its composition repel platelets to minimize and/or eliminate thrombogenesis.

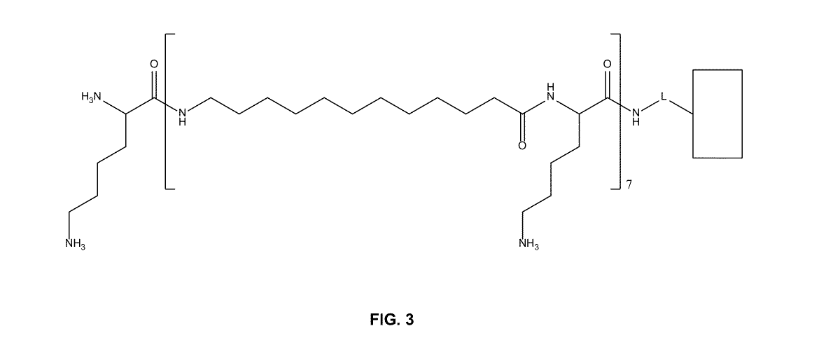

In some embodiments, the capture matrix comprises a peptide-mimetic compound such as a resin bound oligo-acyl-lysine (ROAK), e.g., as shown in FIG. 3. FIG. 3 shows a ROAK having 7 monomer units and a repeating carbon chain component having 12 carbons. The technology is not limited to the particular embodiments shown in FIG. 3. For example, in other embodiments, a ROAK has fewer or more monomer units than 7 and the acyl component has more or fewer than 12 carbons. A number of ROAK compounds and related compounds (such as poly-lysines) are described in, e.g., Rotem et al. (2010) “Bacterial Capture by Peptide-Mimetic Oligoacyllysine Surfaces” Applied and Environmental Microbiology 76: 3301, which is incorporated herein by reference.

In some embodiments, the capture matrix comprises an antibody, antibody fragment, aptamer, or other biomolecule for specific molecular recognition of an analyte to bind and capture the analyte. In some embodiments, the capture matrix comprises a submicron (e.g., ˜500 nm) superparamagnetic anion-exchanger (SiMAG-DEAE).

In some embodiments, the capture matrix comprises a capture oligonucleotide to capture specific nucleic acids present in the blood. In some embodiments, the capture matrix comprises a substance for the non-specific capture of nucleic acids (e.g., glass, silica).

In some embodiments, the capture matrix comprise a component to capture analytes that are proteins, hormones, lipids, small molecules (e.g., drugs), and/or other biological molecules by art-recognized affinity and/or capture techniques.

In some embodiments, the capture matrix comprises a lectin, a carbohydrate, and/or a polysaccharide.

### Analytes

The device is not limited in the analyte captured by the device and, in particular, by the capture matrix. In some embodiments, the analyte is a biological organism associated with a disease (e.g., an infectious disease), e.g., a disease that a patient has or is suspected of having. The organism may be a bacterium, a eukaryote, or an archaeon. The analyte is, in some embodiments, a virus, virion, or similar nucleic acid based infectious particle. In some embodiments, the analyte is a prion or similar proteinaeceous particle. In some embodiments, the analyte is a biomolecule (e.g., a nucleic acid, polypeptide, lipid, carbohydrate, hormone, cofactor, polysaccharide, toxin, metabolite, biomarker) associated with a disease state, e.g., an infectious disease, genetic based disease (e.g., a cancer), etc. In some embodiments, the analyte is a drug or other bioactive substance. In some embodiments, the analyte is a lipid, a sugar, a hormone, a cell, cell component, vesicle, exosome, organelle, ion, salt, antibody, or other biological entity associated with a patient's health, well-being, status, and/or disease state. It is contemplated that embodiments are not limited to detecting one type or class of analyte, but that any combination of organisms, analyte types, or instances is encompassed by embodiments of the technology.

### Methods

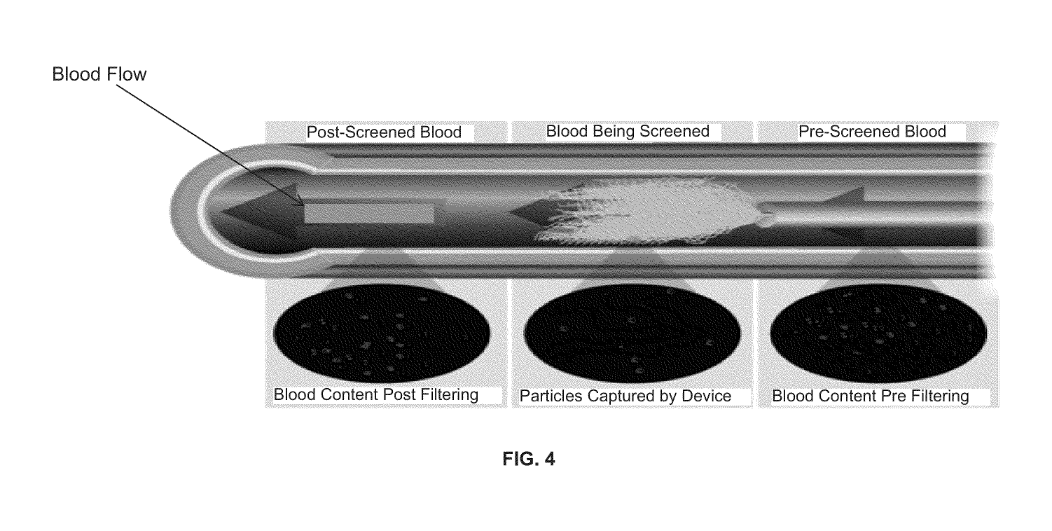

The technology provides embodiments of methods associated with the device embodiments described herein. The technology provides embodiments of methods associated with the device embodiments described herein. For example, the device is used in methods having one or more of the following steps performed in any order: providing a device according to a device embodiment described herein, inserting the device within the vasculature of a patient (e.g., a blood vessel such as a vein or an artery, e.g., the radial/ulnar vasculature), diverting at least a portion of a patient's blood flow through the device, contacting the capture matrix with patient blood (˜5 milliliters up to 5× the patient's total blood volume), attaching a removable cartridge comprising a capture matrix, flowing blood through the device for a length of time sufficient to collect one or more analyte(s) on the capture matrix of the device (e.g., for 5, 10, 15, 20, 30, 45 minutes; 1, 2, 3, 4, 5, 6, 7, 8, 9, 10, 11, 12, 13, 14, 15, 16, 17, 18, 19, 20, 21, 22, 23, 24, or more hours), removing all or part of the capture matrix from the device (e.g., as a part of a removable cartridge), replacing a removable cartridge, recovering analyte from the capture matrix, removing the device from the patient, testing an analyte, reporting results of analysis of the analyte, and administering a drug. Some methods comprise preparing a sample from a recovered analyte (e.g., lysing cells, concentrating, extracting, purifying, isolating, washing, diluting, etc.). Accordingly, the methods provide for the filtering of blood, e.g., to capture analytes from the blood (see FIG. 4).

In embodiments related to an extracorporeal device, methods comprise inserting a first tip and a second tip into the circulation of a patient (e.g., a blood vessel such as a vein or an artery, e.g., the radial/ulnar vasculature), diverting at least a portion of a patient's blood flow through the device, contacting the capture matrix with patient blood (˜5 milliliters up to 5× the patient's total blood volume), attaching a removable cartridge comprising a capture matrix, flowing blood through the device for a length of time sufficient to collect one or more analyte(s) on the capture matrix of the device (e.g., for 5, 10, 15, 20, 30, 45 minutes; 1, 2, 3, 4, 5, 6, 7, 8, 9, 10, 11, 12, 13, 14, 15, 16, 17, 18, 19, 20, 21, 22, 23, 24, or more hours), removing all or part of the capture matrix from the device (e.g., as a part of a removable cartridge), replacing a removable cartridge, recovering analyte from the capture matrix, removing the device from the patient, testing an analyte, reporting results of analysis of the analyte, and administering a drug. Some methods comprise preparing a sample from a recovered analyte (e.g., lysing cells, concentrating, extracting, purifying, isolating, washing, diluting, etc.). Accordingly, the methods provide for the filtering of blood, e.g., to capture analytes from the blood (see FIG. 4).

In some embodiments, the captured analyte finds use in a downstream use such as polymerase chain reaction, culturing, mass spectrometry, nucleic acid sequencing, immuno-assay (e.g., using antibodies, e.g., fluorescently labeled antibodies, e.g., ELISA and related techniques), hybridization assay (e.g., with a probe, e.g., a fluorescent probe), spectrometry (e.g., UV-visible), fluorimetry, nuclear magnetic resonance techniques, infra-red spectrometry, microscopy, toxicology (e.g., in an animal model), etc. Such downstream uses may provide an analysis of the captured analyte, e.g., characterizing the analyte qualitatively and/or quantitatively.

### Kits

Some embodiments of the technology provide a kit for the analysis of a patient blood sample. For example, some kits comprise a short-term vascular implant or an extracorporeal device as described herein and a reagent for processing a captured analyte. Examples of reagents included in kit embodiments include, but are not limited to, lysis solution (e.g., for lysing cells such as microbial cells), wash solution, buffers, salt solutions, chelators, primers for PCR, probes, preservatives, stabilizers, diluents, stains, sterilizing solutions, solutions for mass spectrometry analysis, and/or solutions for immunological analysis of a captured analyte. Solutions are provided, in some embodiments, in a lyophilized form and, in some embodiments, as liquids. In some embodiments, solutions are provided in a concentrated form. Solutions may be provided in any suitable vessel, e.g., a tube, ampule, bottle, jar, can, box, vial, bag, etc. In some embodiments, the extracorporeal device is provided in a kit as a separate tubing component and capture matrix (e.g., either as a component of a cartridge or not as a component as a cartridge).

Kit embodiments comprise one or more capture matrices for capture of one or more analytes from patient blood. In some embodiments, the kit comprises a cartridge comprising a capture matrix. Cartridges are interchangeable with the device (e.g., with the tubing component of the device) and provide a modular technology for the capture of one or more analytes. In some embodiments, a cartridge comprises one capture matrix and in some embodiments a cartridge comprises more than one capture matrix (e.g., for the capture of more than one analyte by a single cartridge). In some embodiments, kits comprise more than one cartridge (e.g., a plurality of cartridges) that are the same type (e.g., that comprise the same capture matrix). In some embodiments, kits comprise more than one cartridge (e.g., a plurality of cartridges) that are a different type (e.g., that comprise a different capture matrix). In some embodiments, kits comprise more than one cartridge (e.g., a plurality of cartridges) that comprise the same type of capture matrix but that differ in the amount and/or analyte binding capacity of the capture matrix.

In some embodiments, kits comprise sample tubes or vessels for collection, preparation, and/or analysis of a collected analyte.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. An device for collecting an analyte from a patient's blood flow, the device comprising:
a) a capture matrix; and
b) a tubing component.

2. The device of claim 1 wherein the capture matrix is removable from the device.

3. The device of claim 1 further comprising a removable cartridge comprising the capture matrix.

4. The device of claim 1 wherein the capture matrix has a higher affinity for the analyte relative a component of the patient's blood.

5. The device of claim 1 wherein the analyte is a pathogen.

6. The device of claim 1 wherein the analyte is a nucleic acid.

7. The device of claim 1 further comprising the analyte.

8. The device of claim 1 wherein the capture matrix comprises a resin bound oligo-acyl-lysine.

9. The device of claim 1 further comprising:
c) a first venipuncture tip; and
d) a second venipuncture tip.

10. The device of claim 1 further comprising a biologically active compound.

11. The device of claim 1 further comprising an anti-thrombogenic agent.

12. The device of claim 1 further comprising a filter, valve, cartridge interface, or Y-connector.

13. The device of claim 1 configured to promote laminar flow of blood through the device.

14. A method for capturing an analyte from a patient's blood, the method comprising:
a) diverting patient blood through a device according to any one of claims 1-13; and
b) capturing an analyte from the patient's blood on the capture matrix.

15. The method of claim 14 further comprising recovering the analyte from the capture matrix.

16. The method of claim 14 wherein the diverting is over a time of 5 minutes to 12 hours.

17. The method of claim 14 wherein the capture matrix is exposed to more than 5 milliliters of patient blood, more than 500 milliliters of patient blood, more than 1 liter of patient blood, more than 5 liters of patient blood, more than 10 liters of patient blood, more than 20 liters of patient blood, more than 0.5× the total blood volume of the patient, more than lx the total blood volume of the patient, more than 2× the total blood volume of the patient, more than 3× the total blood volume of the patient, more than 4× the total blood volume of the patient, or more than 5× the total blood volume of the patient.

18. The method of claim 14 further comprising analyzing the analyte.

19. The method of claim 18 further comprising administering a drug to the patient based on the result of the analyzing.

20. A kit for the analysis of a patient blood sample for an analyte, the kit comprising:
a) a device according to any one of claims 1-13; and
b) a reagent for processing a captured analyte.

21. The kit of claim 20 comprising reagents for lysing bacterial cells to produce a cell lysate and preparing nucleic acid from the cell lysate.

22. The kit of claim 20 comprising a cartridge comprising a capture matrix.

23. The kit of claim 20 further comprising reagents for polymerase chain reaction analysis, mass spectrometry analysis, or immunological analysis of a captured analyte.
